(19) United States
(12) Patent Application Publication
(10) Pub. No.: US 2009/0081260 A1
(43) Pub. Date: Mar. 26, 2009

(54) NATURALLY RESORBABLE POLYACRYLAMIDE GEL, PRODUCTION PROCESS AND USES

(75) Inventors: JEAN-LOUIS BISSON, TALENCE (FR); GENEVIEVE BAUTE, SAINT MEDARD D'EYRANS (FR); HELENA CHEMINET, GRADIGNAN (FR)

Correspondence Address: YOUNG & THOMPSON, 209 Madison Street, Suite 500, ALEXANDRIA, VA, 22314, US

(73) Assignee: PROCYTECH (SOCIETE PAR ACTIONS SIMPLIFIEE), MARTILLAC CEDEX (FR)

(21) Appl. No.: 11/869,761

(22) Filed: Oct. 10, 2007

(30) Foreign Application Priority Data
Sep. 20, 2007 (FR) 07/57697

(51) Int. Cl. A61L 27/16; A61K 8/02; A61K 8/81

(52) U.S. Cl. 424/401; 424/426; 424/78.35; 523/113

(57) ABSTRACT

A biodegradable polyacrylamide gel that is obtained from acrylamide monomers that are connected to one another by at least one amino acid includes at least one acryloylated amine or acryloylated amide group, or by at least one amino acid oligopeptide that comprises at least one acryloylated amine or acryloylated amide group. The process for synthesis of this gel and its uses are also disclosed.

This invention relates to a naturally resorbable biocompatible gel that can be used as an implant and optionally can be injected in human or animal tissues for cosmetic and/or therapeutic purposes.

The invention also relates to a process for synthesis of this gel as well as its uses.

Nowadays, injectable implants are very desirable for numerous applications, such as, for example, tissue reconstruction, treatment of urinary incontinence or else wrinkle reduction.

In a general way, it is desired to be able to modify the appearance of a portion of the body by carrying out plastic surgery or by reconstructing this portion of the body following an injury, an illness or for a cosmetic purpose.

Among the biomaterials that are currently used, degradable injectable products, such as collagens or hyaluronic acids, and non-degradable products, such as polymethyl methacrylate, acrylic hydrogels or else polyacrylamides, are known. It is possible to cite, for example, injectable implants that comprise porous microparticles, in particular described in the patent applications EP-1,311,111 and FR-2,785,811.

The known biodegradable, injectable products, such as the hyaluronic acid esters that are described in the U.S. Pat. No. 4,957,744, are widely used to remodel certain skin imperfections, in particular for filling in wrinkles or hollows. They generally have a satisfactory stability, but with a service life of between 3 and 18 months, their effect is limited over time, which calls for a frequent renewal of injections.

The other products, for their part, are not resorbable and can cause long-term secondary effects, such as the appearance of sometimes painful and always unsightly granulomas. In addition, the known polyacrylamides, produced from acrylamide monomers and pluri-functional acrylamide derivatives, are totally inert insoluble, and hydrophilic, which does not facilitate their use.

There is therefore a need for a product that can replace the extracellular matrix of human or animal tissues, at the same time biocompatible, biodegradable, easy to use, without a health risk and of which it is possible to monitor the resorbability and the period of effectiveness.

This is the purpose of this invention, which proposes a naturally resorbable original polyacrylamide gel.

In particular, this invention aims at a biodegradable polyacrylamide gel that is obtained from acrylamide monomers that are connected to one another by at least one amino acid that comprises at least one acryloylated amine or acryloylated amide group, or by at least one amino acid oligopeptide that comprises at least one acryloylated amine or acryloylated amide group.

Such a gel is capable of acting as a substitute for the extracellular matrix. Advantageously, it is naturally resorbable, and its service life can be modulated with precision.

Other characteristics and advantages will emerge from the following description of the invention.

The polyacrylamide gel according to the invention consists of acrylamide monomers that are connected to one another by at least one amino acid that comprises at least one acryloylated amine or acryloylated amide group, or by at least one amino acid oligopeptide that comprises at least one acryloylated amine or acryloylated amide group.

According to a first variant, the polyacrylamide gel according to the invention corresponds to the following formula:

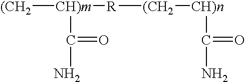

with n and m representing positive integers and R, binding agent of the acrylamide monomers, showing either an amino acid that comprises an acryloylated amine or acryloylated amide group, or an amino acid oligopeptide that comprises an acryloylated amide or an acryloylated amine group.

According to another variant, the polyacrylamide gel according to the invention corresponds to the following formulation:

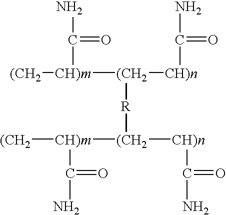

with n and m representing positive integers and R, binding agent of the acrylamide monomers, representing either an amino acid that comprises two acryloylated amine or acryloylated amide groups, or an amino acid oligopeptide that comprises two acryloylated amide or acryloylated amine groups.

The acryloylated amino acids that are used for the production of the gel according to the invention can be obtained by reacting amino acids that comprise at least one amine or amide group with at least one acryloyl chloride or one methacryloyl chloride.

According to a preferred embodiment of the invention, the acryloylated amino acids are obtained by the Schotten-Baumann reaction by making an amino acid that comprises at least one amine or amide group react with an acryloyl chloride, according to the following reaction diagram:

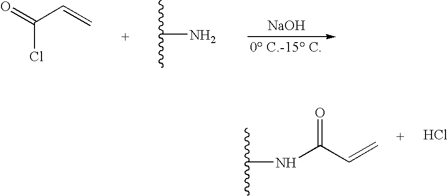

The amino acids that are used can be natural or synthetic. By way of example, it is possible to cite lysine, glutamine, asparagine or arginine.

According to a particular embodiment, the binding agents of the acrylamide monomers are oligopeptides that are formed from 2 to 6 amino acids that comprise at least one acryloylated amine or acryloylated amide group.

Preferably, the gel is a hydrogel that consists of 90% to 99.5% water and polyacrylamide that is obtained from acrylamide monomers that are connected to one another by at least one amino acid that comprises at least one acryloylated amine or acryloylated amide group, or by at least one amino acid oligopeptide that comprises at least one acryloylated amine or acryloylated amide group.

The gel according to the invention can be used in particular as an injectable implant in man or animal.

Advantageously, whereby the acryloylated amino acids or the acryloylated oligopeptides are biodegradable, the gel according to the invention is naturally resorbable when it is injected into human or animal tissue. It is gradually colonized then replaced by substitution of the extracellular matrix during its resorption.

Based on the binding agents that are used (amino acids that are different in number and/or in nature, oligopeptides that are different in number and/or in nature), the service life of the gel in vivo can be modulated with precision. According to their sensitivity to the tissue enzymes, the binding agents are degraded more or less quickly, releasing short polyacrylamide chains with a molecular weight that is low enough to be soluble. These chains are entrained in the interstitial liquids and then excreted just as they are into the bile ducts. The gradual elimination of these polyacrylamide fragments gradually reduces the mass of the gel until it disappears completely.

The resorption of the gel according to the invention is therefore done by gradual elimination of short and soluble polyacrylamide chains.

According to another advantage, the gel according to the invention can be charged positively, for example by adding a molecule such as the DADMA. The circulating molecules are then drawn off inside the gel, which facilitates its role as substitute in the extracellular matrix.

The polyacrylamide gel according to the invention can be obtained by the implementation of a process that comprises in particular the following stages:
Synthesis of acryloylated amino acids or acryloylated amino acid oligopeptides, starting from amino acids that comprise at least one amine or amide group,
Purification of the acryloylated amino acids or acryloylated amino acid oligopeptides that are obtained to eliminate the organic products and the amino acids that have not reacted, and
Synthesis of the polyacrylamide gel by reacting acrylamide monomers with acryloylated amino acids or purified acryloylated amino acid oligopeptides.

Preferably, the synthesis of the acryloylated amino acids consists in reacting amino acids that comprise at least one amine or amide group with at least one acryloyl chloride or one methacryloyl chloride.

Even more preferably, the acryloylated amino acids are obtained by the Schotten-Baumann reaction by reacting an amino acid that comprises at least one amine or amide group with an acryloyl chloride.

According to an embodiment, the synthesis of the acryloylated amino acids is carried out according to the following operating procedure:
An aqueous medium is used with sodium hydroxide, containing amino acids that comprise at least one amide or amino group,
The medium is put under vacuum to expel the air, then put under an inert atmosphere at a temperature of between −5° C. and 15° C., to prevent the hydrolysis of the acryloyl chloride that occurs according to the reaction diagram below

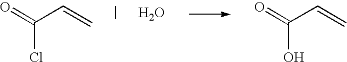

Acryloyl chloride is gradually added to the medium that contains the amino acids, and
The reaction medium is mixed.

The purification stage of the acryloylated amino acids or acryloylated amino acid oligopeptides that are obtained can comprise an extraction by ethyl acetate, in particular for eliminating the excess acryloyl or methacryloyl chloride.

Preferably, the purification of the acryloylated amino acids or the acryloylated amino acid oligopeptides that are obtained is carried out by the implementation of the following stages:
Acidification of the reaction medium by hydrochloric acid, and
Extraction by ethyl acetate.

Finally, the synthesis of the polyacrylamide gel according to the invention is performed by reacting acrylamide monomers with acryloylated amino acids or purified acryloylated amino acid oligopeptides.

Advantageously, the precise monitoring of the parameters of the reaction makes it possible, for an identical quali-quantitative composition, to obtain gels with different viscosities and different biological half-life periods. It is thus possible to envisage different applications.

In particular, the gel according to the invention can be used:
For tissue enhancement,
For viscosupplementation,
As a matrix for cellular culture,
For the prevention of adhesions,
For tissue regeneration,
For tissue repair, and
For improving the host-implant interface.

The polyacrylamide gel according to the invention can be used as an implant to increase the tissue volumes, in particular in cosmetic surgery, in urology, in gastroenterology, in rheumatology and in gynecology.

In cosmetic surgery, the gel according to the invention is particularly useful for correcting wrinkles and lipodystrophies.

In urology, the gel according to the invention can be used for treating urinary stress incontinence or vesicourethral reflux. The urinary stress incontinence in women is due to a weakness of the urethral sphincter and/or a cervicourethral hypermobility. The increase in the volume of the sub-mucosal tissue by injection of the gel according to the invention causes the urethral walls to pull together and therefore produces better closing, which makes it possible to restore urinary continence.

In gastroenterology, proctology, the gel according to the invention is useful for treating gastro-esophageal reflux or anal incontinence. Actually, peri-anal implantation of the gel according to the invention makes it possible to compensate for resting anal hypotonia in patients who suffer from passive fecal incontinence. Furthermore, the injection or the implantation of the gel according to the invention in the gastro-esophageal sphincter causes a reduction of the opening or a thickening and a sphincter fibrosis thus reconstituting an anti-reflux barrier.

In rheumatology and in sports medicine, the gel according to the invention makes it possible in particular to treat talalgia, chronic heel pain, by reestablishing the thickness and the mechanical properties of the pad of atrophied heels.

In surgical gynecology, the volumizing action of the gel according to the invention can be used for clitoral reconstruction, for vulvoplasties and vaginoplasties and for vaginal rejuvenation.

According to a second aspect, the polyacrylamide gel according to the invention can be used for viscosupplementation, in particular for treating osteoarthritis. The intra-articular administration of the gel according to the invention makes it possible to reduce the pain and to improve the articular function by contributing to stimulating the natural process of cartilage recovery.

According to another aspect, it is possible to use the gel according to the invention as a matrix for cellular culture, with applications in particular in cosmetic surgery, dermatology, rheumatology and gastroenterology. Actually, the resorbable gel according to the invention is a good three-dimensional substrate for supporting the growth of various types of cells.

In cosmetic surgery, it is possible to cite applications for capillary cloning or for implants for filling in wrinkles or hollows.

In dermatology, the gel can be used for healing chronic wounds: as a matrix, it makes possible the tangential development of the process of healing and the prevention of budding in the case of hypertrophic healing.

In rheumatology and orthopedics, the use of the gel according to the invention as a matrix for the cellular culture is particularly suitable for the repair of the cartilage by chondroinduction.

Relative to the applications of the gel as a three-dimensional substrate for the cellular growth in gastroenterology, it is possible to cite in particular the use in an artificial pancreas to protect the pancreatic cells from attacks of the immune system and therefore from rejection phenomena.

According to a fourth aspect, the polyacrylamide gel according to the invention is useful for the prevention of adhesions, in particular in visceral and thoracic surgery for preventing abdominal adhesions after pelvic surgery or an appendectomy. The gel is able to bond with a target tissue to form a flexible and adhesive barrier.

According to a still different aspect, it is possible to use the gel according to the invention for tissue regeneration, in particular in rheumatology, orthopedics, traumatology, in neurosurgery, in dermatology, in gynecology and in orthodontics.

In rheumatology, orthopedics, traumatology, the gel is useful in particular for the restoration of collapsed or degenerated intervertebral disks, or for the stimulation of bone formation during bone strengthening.

In neurosurgery, the applications essentially relate to the regeneration of damaged neural pathways in the central nervous system.

In dermatology, the gel according to the invention can be used for the management of dry, fibrinous and necrotic wounds, the softening and the separation of the dry necrosis in different types of wounds, or else the healing of chronic wounds.

In gynecology, the use of the gel according to the invention relates to the treatment of the vulvovaginal drying.

In orthodontics, the cellular regeneration using the gel according to the invention makes it possible to treat inflammatory conditions such as gingivitis, bleeding, gingival hollows or gingival pockets, or traumatic conditions.

According to a sixth aspect, the gel according to the invention is useful for tissue reparation, in particular in orthopedic surgery of the vertebral column to replace the dorsal gray column and to preserve or restore an intervertebral disk height, a mobility and a mechanical function of the lumbar vertebral column that are optimal.

Finally, the gel according to the invention can be used to improve the biocompatibility of an implant, such as, for example, a vascular prosthesis, for the purpose of reducing the reactions to foreign bodies.

Of course, the invention obviously is not limited to the examples of synthesis processes and to uses that are presented and described above, but on the contrary covers all the variants.

1. Biodegradable polyacrylamide gel that is obtained from acylamide monomers that are connected to one another by at least one amino acid that comprises at least one acryloylated amine or acryloylated amide group, or by at least one amino acid oligopeptide that comprises at least one acryloylated amine or acryloylated amide group.

2. Polyacrylamide gel according to claim 1, wherein the amino acids are obtained by reaction of amino acids comprising at least one acryloylated amine or acryloylated amide group, with at least one acryloyl chloride or one methacryloyl chloride.

3. Polyacrylamide gel according to claim 2, wherein the amino acids are selected from among lysine, glutamine, asparagine and arginine.

4. Polyacrylamide gel according to claim 1, wherein the acrylamide monomers are connected to one another by at least one oligopeptide that is formed from 2 to 6 amino acids.

5. Polyacrylamide gel according to claim 1, wherein it is a hydrogel that constitutes between 90% and 99.5% water.

6. Process for obtaining a polyacrylamide gel according to any of the preceding claims, wherein it comprises the following stages:
Synthesis of acryloylated amino acids or acryloylated amino acid oligopeptides, starting from amino acids that comprise at least one amine or amide group,
Purification of the acryloylated amino acids or acryloylated amino acid oligopeptides that are obtained to eliminate the organic products and the amino acids that have not reacted, and
Synthesis of the polyacrylamide gel by reacting acrylamide monomers with acryloylated amino acids or purified acryloylated amino acid oligopeptides.

7. Process for obtaining a polyacrylamide gel according to claim 6, wherein the synthesis of the acryloylated amino acids consists in reacting amino acids that comprise at least one amine or amide group with at least one acryloyl chloride or one methacryloyl chloride.

8. Process for obtaining a polyacrylamide
gel according to claim 7, wherein the purification comprises a stage for extraction via ethyl acetate to eliminate the excess acryloyl chloride or methacryloyl chloride.

9. Use of a polyacrylamide gel according to claim 1
as a naturally resorbable implant, substitute of the extracellular matrix.

10. Use of a polyacrylamide gel according to claim 1,
for the enhancement of human or animal tissues, in particular in cosmetic surgery to correct wrinkles and lipodystrophies.

11. Use of a polyacrylamide gel according to claim 1, for tissue regeneration, for tissue repair or as a matrix for cellular culture.
